(19) United States
(12) Patent Application Publication
(10) Pub. No.: US 2017/0266213 A1
(43) Pub. Date: Sep. 21, 2017

(54) USE OF BETA-NICOTINAMIDE MONONUCLEOTIDE IN PREPARATION OF ANTI-AGING DRUGS OR HEALTH-CARE PRODUCTS

(71) Applicant: BONTAC BIO-ENGINEERING (SHENZHEN) CO., LTD, Shenzhen (CN)

(72) Inventors: Rongzhao FU, Shenzhen (CN); Qi ZHANG, Shenzhen (CN)

(21) Appl. No.: 15/310,371

(22) PCT Filed: Dec. 3, 2015
(86) PCT No.: PCT/CN2015/096334

§ 371 Date: Nov. 10, 2016

(30) Foreign Application Priority Data
Mar. 16, 2015 (CN) 201510113652.0

(51) Int. Cl. A61K 31/706; A61K 9/28

(52) U.S. Cl. CPC A61K 31/706; A61K 9/28

(57) ABSTRACT

Disclosed is use of β-nicotinamide mononucleotide in the preparation of anti-aging drugs or health-care products. A single dose of the β-nicotinamide mononucleotide is 1-500 mg/Kg body weight/day, and the drug or health-care product is in the form of tablets, capsules, granules, aqueous solutions, enteric-coated preparations or injections.

### BACKGROUND

Technical Field

The present invention relates to the technical field of use of NMN, and particularly to use of β-nicotinamide mononucleotide in the preparation of anti-aging drugs or health-care products.

Related Art

With the continuous improvement of people's living standards, the desire to pursue health and longevity becomes increasingly strong. However, aging is an inevitable tendency of any life process, and a biological lay independent of man's will. However, it is possible to achieve the maximum life span given by the nature by slowing down the rate of aging. The anti-aging drugs or health-care products available in China are mostly Chinese herbal medicine compound preparations. For example, Qi Jianhua et al (issued Patent No. CN102247524B, 2012) find that a total steroidal saponin extract has an anti-aging effect. Due to the complex ingredients in the Chinese herbal medicine compound preparations, the efficacy and toxic effect are questioned. Moreover, some additional anti-aging drugs or health-care products either have poor efficacy or high cost.

Therefore, there is still a need for improvement and development in the prior art.

### SUMMARY

### Technical Problem

In view of the disadvantages existing in the prior art, an objective of the present invention is to provide use of β-nicotinamide mononucleotide in the preparation of anti-aging drugs or health-care products, so as to solve the problem of poor efficacy, high cost, and presence of toxic adverse effect of existing anti-aging drugs or health-care products.

### Solution to the Problem

### Technical Solution

The technical solution of the present invention is as follows.

Use of β-nicotinamide mononucleotide in the preparation of anti-aging drugs or health-care products is provided, where the β-nicotinamide mononucleotide is used in the preparation of anti-aging drugs or health-care products.

In the use of β-nicotinamide mononucleotide in the preparation of anti-aging drugs or health-care products, a single dose of the β-nicotinamide mononucleotide is 1-500 mg/Kg body weight/day.

In the use of β-nicotinamide mononucleotide in the preparation of anti-aging drugs or health-care products, a single dose of the β-nicotinamide mononucleotide is 1-20 mg/Kg body weight/day.

In the use of β-nicotinamide mononucleotide in the preparation of anti-aging drugs or health-care products, the drug or health-care product is in the form of tablets, capsules, granules, aqueous solutions, enteric-coated preparations, or injections.

In the use of β-nicotinamide mononucleotide in the preparation of anti-aging drugs or health-care products, the drug or health-care product is in the form of tablets.

In the use of β-nicotinamide mononucleotide in the preparation of anti-aging drugs or health-care products, the tablets are enteric-coated tablets.

### Beneficial Effect of the Invention

### Beneficial Effect

In the present invention, β-nicotinamide mononucleotide (NMN) is used as an active ingredient in the preparation of anti-aging drugs or health-care products. Specific dosage depends on severity of the disease, route of administration, and other relevant factors. NMN is a substance present in cells of organisms, which is highly safe as a drug or health-care product. Moreover, the NMN is a monomeric molecule and has obvious and stable efficacy.

### DETAILED DESCRIPTION

The present invention provides use of β-nicotinamide mononucleotide in the preparation of anti-aging drugs or health-care products. To make the objective, technical solution, and effect of the present invention clearer, the present invention is described in further detail below. It should be understood that specific embodiments described herein are merely illustrative of, instead of limiting the present invention.

β-nicotinamide mononucleotide (NMN) is a biochemical substance present in cells of organism, and is one of the precursors for synthesizing nicotinamide adenine dinucleotide (also known as coenzyme I) in cells.

Nicotinamide adenine dinucleotide is a fundamental redox coenzyme, which is key to both the respiration and the photosynthesis. At present, β-nicotinamide mononucleotide (NMN) can be produced through fermentation with yeasts or chemical synthesis, or produced enzymatically in vitro. The β-nicotinamide mononucleotide used in the present invention is produced enzymatically in vitro, which contains no organic solvent residue, has no problem of chirality, is an isoform of the β-NMN in organisms, and is cost effective.

The present invention provides use of NMN in the preparation of anti-aging drugs or health-care products, where the NMN is used in the preparation of anti-aging drugs.

A single dose is 1-500 mg/Kg body weight/day, and preferably 1-20 mg/Kg body weight/day, for example 10 mg/Kg body weight/day. Specific dosage depends on severity of the disease, route of administration, and other relevant factors.

In the present invention, the drug or health-care product is in the form of tablets, capsules, granules, aqueous solutions, enteric-coated preparations, or injections.

The drug or health-care product is in the form of tablets, and preferably enteric-coated tablets.

In the present invention, NMN is used as part of the active ingredient in the preparation of anti-aging drugs or health-care products, and preferably as the sole active ingredient in the preparation of anti-aging drugs or health-care products.

### EXAMPLE 1

### Use of β-Nicotinamide Mononucleotide in the Preparation of Anti-Aging Drugs or Health-Care Products

The NMN, together with pharmaceutically acceptable adjuvants, was prepared into various dosage forms by using conventional methods in the art of medicines and health-care products. When provided for oral administration, the NMN may be prepared into conventional solid preparations, for example, tablets, powders or capsules; and when provided for injection, the NMN may be prepared into injectable solutions.

(1) Weighing: 10 g NMN, food grade;

40 g D-mannitol; 20 g microcrystalline cellulose were weighed.

(2) Mixing uniformly and tabletting: 0.5 g magnesium stearate was added, mixed uniformly and tabletted.

(3) Coating: The tablets were coated with 2.5 g seal coating and 8 g enteric coating.

(4) : The tablets were packaged.

### EXAMPLE 2

### Pharmacodynamics Experiment

### Materials and Methods

1.1 Experimental material: β-nicotinamide mononucleotide (NMN)

1.2 Experimental animal: Kunming mice (aged about 52 weeks)

1.3 Administration method:

Blank group: fed on full diet;

Treatment group: fed on full diet supplemented with NMN (containing adjuvants) fully dissolved in water, where a single dose of NMN is 20 mg/Kg body weight/day;

Control group: fed on full diet supplemented with adjuvants equivalent to those in the Treatment group;

Mouse survival test: 120 mice aged 52 weeks were assigned to two groups (that is, a blank control group and a treatment group) at random, each group having 40 animals.

Experimental method: see Function Evaluation procedures and Specification of test methods for health food—(2003 Edition, the Ministry of Health).

The result of mouse survival test is shown in Table 1

TABLE 1

| | Morbility | Morbility | Morbility | Morbility | Morbility |
|---|---|---|---|---|---|
| Experiment | over 100 | over 120 | over 140 | over 160 | over 180 |
| group | weeks (%) | weeks (%) | weeks (%) | weeks (%) | weeks (%) |
| Blank group | 45 | 84 | 100 | | |
| Control | 44 | 86 | 100 | | |
| group | | | | | |
| Treatment | 38 | 52 | 61 | 74 | 100 |
| group | | | | | |

It can be seen from Table 1 that the NMN composition prepared in the present invention can extend the lifespan of mice by about 29%.

In summary, β-nicotinamide mononucleotide is used as active ingredients in the preparation of anti-aging drugs or health-care products in the present invention. The composition containing NMN can obviously extend the lifespan of mice.

It should be understood that the use of the present invention is not limited to those exemplified above. Modifications or changes may be made by those ordinarily skilled in the art based on the disclosure above, which are all contemplated in the protection scope of the present invention as defined by claims below.

What is claimed is:

1. Use of β-nicotinamide mononucleotide in the preparation of anti-aging drugs or health-care products, wherein the β-nicotinamide mononucleotide is used in the preparation of anti-aging drugs or health-care products.

2. The use of β-nicotinamide mononucleotide in the preparation of anti-aging drugs or health-care products according to claim 1, wherein a single dose of the β-nicotinamide mononucleotide is 1-500 mg/Kg body weight/day.

3. The use of β-nicotinamide mononucleotide in the preparation of anti-aging drugs or health-care products according to claim 2, wherein a single dose of the β-nicotinamide mononucleotide is 1-20 mg/Kg body weight/day.

4. The use of β-nicotinamide mononucleotide in the preparation of anti-aging drugs or health-care products according to claim 1, wherein the drug or health-care product is in the form of tablets, capsules, granules, aqueous solutions, enteric-coated preparations, or injections.

5. The use of β-nicotinamide mononucleotide in the preparation of anti-aging drugs or health-care products according to claim 4, wherein the drug or health-care product is in the form of tablets.

6. The use of β-nicotinamide mononucleotide in the preparation of anti-aging drugs or health-care products according to claim 5, wherein the tablets are enteric-coated tablets.
